(19) United States
(12) Patent Application Publication
(10) Pub. No.: US 2007/0160648 A1
(43) Pub. Date: Jul. 12, 2007

(54) Methods for treating otic disorders

(75) Inventors: Paul Ashton, Boston, MA (US); Hong Guo, Belmont, MA (US); Thomas J. Smith, Altadena, CA (US)

Correspondence Address: FISH & NEAVE IP GROUP;ROPES & GRAY LLP, ONE INTERNATIONAL PLACE, BOSTON, MA, 02110-2624, US

(73) Assignee: pSivida Inc., Watertown, MA (US)

(21) Appl. No.: 11/710,045

(22) Filed: Feb. 23, 2007

Related U.S. Application Data
(63) Continuation of application No. 10/372,636, filed on Feb. 24, 2003.
(60) Provisional application No. 60/358,831, filed on Feb. 22, 2002.

(51) Int. Cl. A61F 2/02

(52) U.S. Cl. 424/426

(57) ABSTRACT

Loss of hearing can be treated by implanting a sustained-release drug delivery device in the inner ear. The slow delivery of medication from the implanted device to the tissues of the ear, including the inner ear, can treat numerous conditions of the ear while avoiding the side effects associated with systemic administration.

### RELATED APPLICATIONS

This application claims priority of U.S. provisional application No. 60/358/831, filed Feb. 22, 2002, the entire contents of which are incorporated herein by reference.

### FIELD OF THE INVENTION

This invention relates to the fields of pharmaceuticals, drug delivery devices, methods for sustained drug release, and methods for treatment of hearing loss, infections, and other pathological conditions of the middle and inner ear.

### BACKGROUND OF THE INVENTION

1. Chemically-Induced Hearing Loss.

Hearing loss afflicts over ten percent of the population of the United States. Damage to the peripheral auditory system is responsible for a majority of such hearing deficits. In particular, destruction of hair cells and of the primary afferent neurons in the spiral ganglia, which transduce auditory signals from the hair cells to the brain, have been implicated as major causes of hearing impairments.

Agents causing hearing impairment include loud noise, aging, infections, and ototoxic chemicals, among which are aminoglycoside antibiotics and platinum-containing antineoplastic agents such as cisplatin. Ototoxins such as cisplatin and aminoglycoside antibiotics accumulate in cochlear hair cells, and cellular damage to these cells resulting from the accumulation is thought to be the primary reason for chemically-induced hearing loss.

The peripheral auditory system consists of auditory receptors, hair cells in the organ of Corti, and primary auditory neurons, the spiral ganglion neurons in the cochlea. Sprial ganglion neurons (“SGN”) are primary afferent auditory neurons that deliver signals from the peripheral auditory receptors, the hair cells in the organ of Corti, to the brain through the cochlear nerve. The eighth nerve connects the primary auditory neurons in the spiral ganglia to the brain stem. The eighth nerve also connects vestibular ganglion neurons (“VGN”), which are primary afferent sensory neurons responsible for balance and which deliver signals from the utricle, saccule and ampullae of the inner ear to the brain.

The vestibular and auditory systems share many characteristics including peripheral neuronal innervations of hair cells and central projections to the brainstem nuclei. Both of these systems are sensitive to ototoxins that include therapeutic drugs, antineoplastic agents, contaminants in foods or medicines, and environmental and industrial pollutants. Ototoxic drugs include the widely used chemotherapeutic agent cisplatin and its analogs, commonly used aminoglycoside antibiotics, e.g. gentamicin, certain macrolide antibiotics (L. She, et al., 1999, Am. J. Health-Syst. Pharm. 56:380-383), glycopeptide antibiotics such as vancomycin, quinine and its analogs, salicylate and its analogs, and loop diuretics.

The toxic effects of these drugs on auditory cells and spiral ganglion neurons are often the limiting factor in their therapeutic usefulness. For example, the aminoglycoside antibiotics (gentamycins, streptomycins, kanamycins, tobramycins, and the like) are broad-spectrum antimicrobials effective against gram-positive, gram-negative and acid-fast bacteria. They are used primarily to treat infections caused by gram-negative bacteria, often in combination with beta lactams which provide synergistic effects. Advantages to using the aminoglycoside antibiotics include a low incidence of Clostridium difficile diarrhea relative to other antibiotics, and a low risk of allergic reactions. However, the aminoglycosides are known to exhibit serious ototoxicity, especially at higher (and more effective) doses. For example, 25% of patients given one gram of streptomycin daily for 60 to 120 days displayed some vestibular impairment, whereas at two grams per day, the incidence increased to 75%, and some patients suffer permanent damage (see U.S. Pat. No. 5,059,591). For this reason the aminoglycosides are rarely selected by physicians as a first-line therapy, despite their many advantages.

Salicylates, such as aspirin, have long been used for their anti-inflammatory, analgesic, anti-pyretic and anti-thrombotic effects. Unfortunately, salicylates have ototoxic side effects. They often lead to tinnitus (“ringing in the ears”) and temporary hearing loss, and if used at high doses for a prolonged time, hearing impairment can become persistent and irreversible (J. A. Brien, 1993, Drug Safety 9:143-148).

The most effective and frequently used loop diuretics (such as ethacrynic acid, furosemide, and bumetanide) are known to cause ototoxicity. Several less-commonly used loop diuretics also have been experimentally shown to cause ototoxicity; this group includes torsemide, azosemide, ozolinone, indacrinone, and piretanide. Hearing loss associated with loop diuretics is frequently, but not always, reversible.

Ototoxicity is a serious dose-limiting side-effect for cisplatin (cis-diammine-dichloroplatinum(II), CDDP), a widely-used antineoplastic agent that has proven effective on a variety of human cancers including testicular, ovarian, bladder, and head and neck cancers. The toxic side effects of cisplatin (peripheral neuropathies, myelo-suppression, gastrointestinal toxicity, nephrotoxicity, and ototoxicity) are well-known. The routine administration of mannitol, hypertonic saline, and high fluid administration have largely ameliorated cisplatin-induced nephrotoxicity, leaving ototoxicity as the primary dose-limiting factor today. Thus, although an increasing number of cancer patients are surviving modern regimens of chemotherapy, they frequently suffer from cisplatin-induced hearing impairment.

For equivalent inner ear concentrations, cisplatin is the most ototoxic drug known. Generally, cisplatin ototoxicity is irreversible, its onset insidious, and the hearing loss may progress after discontinuation of the protocol. Hearing loss is usually permanent, although partial recovery may occur in some cases.

Cisplatin damages both the auditory and vestibular systems. The primary ototoxic effects of cisplatin appear to occur in the cochlea. Anatomical changes occur in both the stria vascularis and the organ of Corti. The primary histologic findings include dose-related hair cell degeneration and damage to the supporting cells, and at high doses, total collapse of the membranous labyrinth can occur. In the organ of Corti, there is loss of outer and inner hair cells, with a propensity for outer hair cell loss in the basal turn, and alterations in the supporting cells and Reissner's membrane. Softening of the cuticular plate and an increased number of lysosomal bodies in the apical portion of the outer hair cell have also been reported.

The molecular mechanisms underlying these changes are largely unknown. Several potential mechanisms have been described, including impaired deoxyribonucleic acid (DNA), ribonucleic acid (RNA), and protein synthesis; impaired synthesis and degradation of prostaglandins, gangliosides, mucopolysaccharides, and lipids; and disruptions in metabolism and ion transport.

Accordingly, there exists a need for means to prevent, reduce or treat the incidence and/or severity of inner ear disorders and hearing impairments involving inner ear tissue, particularly inner ear hair cells, and optionally, the associated auditory nerves. Of particular interest are those conditions arising as an unwanted side-effect of ototoxic therapeutic drugs including cisplatin and its analogs, aminoglycoside antibiotics, salicylate and its analogs, or loop diuretics. In addition, there exits a need for methods which will allow higher and thus more effective dosing with these ototoxicity-inducing pharmaceutical drugs, while concomitantly preventing or reducing ototoxic effects. What is needed is a method that provides a safe, effective, and prolonged means for prophylactic or curative treatment of hearing impairments related to inner ear tissue damage, loss, or degeneration, particularly ototoxin-induced and particularly involving inner ear hair cells.

2. Noise-Induced Hearing Loss.

Noise-induced hearing loss (NIHL) describes a chronic hearing-impairing disease process that occurs gradually over many years of exposure to less intense noise levels, wherein the damage is to the inner ear, specifically, the cochlea. This type of hearing loss is generally caused by chronic exposure to high intensity continuous noise with superimposed episodic impact or impulse noise. Both an intense sound presented to the ear for a short period of time and a less intense sound that is presented for a longer time period will produce equal damage to the inner ear. The majority of chronic NIHL is due to occupational or industrial exposure. However, a non-occupational form of NIHL, called socioacusis, may result from gunfire, loud music (via concerts or headphones), open vehicles such as motorcycles, snowmobiles or tractors, and power tools to name just a few. Although the hearing damage is often symmetrical, i.e. both ears are affected, there are cases, such as hearing loss due to frequent target shooting, which result asymmetric hearing loss.

Upon exposure to impulse noise, such as an explosive blast, a patient may suffer significant tympanic membrane and middle ear damage. In chronic exposure, which generally occurs at lower intensity levels, middle ear and tympanic membrane damage are unlikely. In noise exposure, the primary and initial damage is generally cochlear, with secondary neural degeneration of the auditory system occurring over time. Noise-induced hearing loss has been reviewed by K. Campbell in “Essential Audiology for Physicians” (1998), San Diego: Singular Publishing Group, Inc.

3. Otitis Media.

Otitis media is an inflammation of the middle ear, most commonly associated with viral or bacterial infection. A relatively high percentage of the population, particularly children, are affected. In children, the disease is most often associated with upper respiratory afflictions which trigger a transudate secretion response in the Eustachian tube and middle ear. Bacteria and viruses migrate from the naso-pharynx to the normally air-filled middle ear via the Eustachian tube, and can cause the Eustachian tube to become blocked, preventing ventilation and drainage of the middle ear. Fluid then accumulates behind the eardrum, causing pain and inflammation.

Otitis media is the most common cause of hearing loss among children. Although otitis media is readily treated with antibiotics and is ordinarily not serious, frequent and/or untreated otitis media may permanently damage a child's hearing. Fluid remaining in the middle ear can cause repeated bouts of acute otitis media, and if the condition becomes chronic it may result in frequent recurrences of acute infections. In the more severe forms of otitis media, purulent exudate, toxins and endogenous anti-microbial enzymes accumulate in the middle ear, which can cause irreparable damage to sensory-neural and sound conducting structures. Damage to the eardrum, the bones of the ear, or the auditory nerves caused by such infections can cause permanent hearing loss. Hearing loss may also result from impairment, damage or destruction of inner ear cochlear hair cells, as damaging substances in the middle ear space gain access to the inner ear via diffusion through the round window membrane.

4. Treatment and Prevention of Otic Disorders.

One hypothesis to account for hearing impairment due to loud noise, age or chemicals points to reactive oxygen species (ROS) as being the causative agents for cochlear hair cell damage. Some free radical scavengers, iron chelators and certain NMDA receptor antagonists have been shown to be otoprotective agents, which are effective in protecting cochlear hair cells from chemically-induced or noise-induced cell death. Accordingly, approaches to treat hearing impairment due to idiopathic sudden sensory hearing loss (ISSHL), noise induced hearing loss (NIHL), or chemically induced hearing loss (CIHL) have included treatment with otoprotective agents, including antioxidants such as aspirin, reduced glutathione, N-methyl-(D)-glucaminedithiocarbamate, (D)-methionine, and iron chelators such as tartrate and maleate. While these compounds have shown efficacy in some animal models of NIHL and CIHL, to date, only D-methionine has been approved for use to prevent or treat hearing impairment. However, the pharmacological profile of (D)-methionine makes it difficult to administer it to patients.

Other treatments for ototoxicity have involved administration of steroids, vitamins or rheologic agents. Other treatments include the use of vasodilators; vascular rheologic agents such as pentoxyfylline; anticoagulants; plasma expanders such as dextran; renograffin or urograffin, and growth factors such as IGF-1 and FGF-2.

Another difficulty in preventing ototoxicity, especially when due to aminoglycoside antibiotics, is that the damage occurs over a period of time that extends well beyond the time during which the ototoxic agent is administered. Aminoglycosides, for example, can be detected in the cochlea months after the last dose of the drug. Any chemotherapy intended to ameliorate ototoxicity must therefore be administered over a considerable period of time.

There is a pressing need for otoprotective agents that prevent, reduce, or otherwise treat hearing impairment due to noise, age or chemicals. These otoprotective agents would be useful in the context of hazards posed by loud noises in certain occupational or recreational activities, injuries arising from exposure to ototoxic chemicals such as occurs in certain chemotherapeutic regimes, or improving quality of life in aging populations experiencing progressive hearing impairment. For instance, the ototoxicity of aminoglycosides has limited the applications of this very important group of antibiotics, and the ototoxicity of cisplatin adds a further burden to those already facing a life-threatening disease. There is a particular need for otoprotective agents that prevent, reduce, or otherwise ameliorate the ototoxic side-effects of aminoglycoside antibiotics or platinum-containing antineoplastic agents, without compromising the in vivo microcidal or anti-tumor properties of these compounds. Where ototoxicity is the dose-limiting side effect of a chemotherapeutic agent, there is also a need for otoprotective agents that would lift the dose limitation, making it possible to administer higher and more effective doses of the chemotherapeutic agent.

Local administration of neurotoxins, such as botulinum toxin, to middle ear muscles has been disclosed as a method of treating tinnitus, cochlear nerve dysfunctions, and Meniere's disease (U.S. Pat. No. 6,265,379). Other treatments include systemic administration of benzodiazepines and topical anesthetics such as lidocaine. Systemic administration of such drugs is associated with severe side-effects, however, and the therapeutic effect is short-lived without repeated administration of the drugs. There is a need for a method of administration of benzodiazepines and local anesthetics to the middle and inner ear that avoids systemic exposure while providing extended therapeutic benefits.

Treatments for ISSHL include administration of vasodilators, such as papaverine, histamine, nicotinic acid, procaine, and niacin; rheologic agents such as pentoxyfylline, heparin and warfarin; anti-inflammatory agents, particularly corticosteroids; antiviral agents such as acyclovir, famciclovir, valacyclovir and amantadine; and diatrizoate meglumine. Here as well, there is a need for a method of providing therapeutic levels of these drugs within the ear, for a prolonged period of time, without exposing the subject's entire system to the drugs and their potential side-effects.

For individuals at high risk for middle ear infections, antibiotics may be systemically administered in a prophylactic manner. Systemic administration of antibiotics to combat or prevent middle ear infection generally involves a prolonged lag time to achieve therapeutic levels within the ear, requires high initial doses in order to achieve such levels, and in some cases may require administration over a very long period of time. Systemic administration of a drug also brings into play pharmacokinetic variables such as rates of absorption, rates of metabolism, and rates of excretion that vary from patient to patient. These drawbacks complicate the ability to obtain and maintain therapeutic levels, and systemic toxicities may preclude the prophylactic use of some antibiotics altogether. There is a need, therefore, for a method of providing therapeutically effective concentrations of antibiotics in the middle and inner ear over a prolonged period of time, without the disadvantages of systemic administration.

### SUMMARY OF THE INVENTION

The invention relates broadly to the treatment of otic disorders by local and sustained administration of appropriate therapeutic agents to the inner ear. More specifically, the present invention relates in one embodiment to the use of otoprotective agents to prevent, reduce, or otherwise treat hearing impairments, particularly those due to ISSHL, CIHL, NIHL, aging, or infection. Of particular interest in the CIHL category are chemotherapeutic drugs, such as aminoglycoside antibiotics, macrolide antibiotics, platinum-containing antineoplastic agents such as cisplatin, certain quinine-like compounds, and ototoxic diuretic drugs such as the loop diuretics.

The present invention relates to the use of otoprotective agents to prevent, reduce, or otherwise treat ototoxicity associated with NIHL, aging or CIHL. In the case of CIHL due to chemotherapeutic agents, the invention relates to the use of otoprotective agents in a manner that does not compromise the efficacy of chemotherapeutic agents.

Accordingly, one aspect of the present invention describes a method for preventing, reducing or otherwise treating NIHL, CIHL, or hearing impairment due to aging by administering to a patient a pharmaceutical dosage of an otoprotective agent, or a pharmaceutically acceptable salt, solvate, clathrate, prodrug, tautomer or a metabolic derivative thereof.

Still further, the present invention provides a method for treating the ototoxic effects currently associated with certain antibiotics, and particularly with the more popular and commonly used aminoglycoside and macrolide antibiotics without sacrificing antimicrobial effectiveness.

Still further, the invention provides a method for treating the ototoxic effects currently associated with certain chemotherapeutics, and particularly with the more popular and commonly used cisplatin chemotherapeutics without sacrificing the antineoplastic effectiveness of cisplatin or its analogs.

Still further, the present invention provides a method for treating the ototoxic effects currently associated with certain quinines and quinidines without sacrificing their effectiveness. The adverse side effects of quinine and quinidine are similar, and have been given the name “cinchonism,” deriving from the fact that quinine is obtained from the bark of the cinchona tree. These side effects include disturbances of hearing, including tinnitus, deafness, and vertigo.

Another object of the invention is the new method of treatment of patients, particularly children, having purulent otitis or other chronic ear infections, comprising the use of a sustained release drug device described herein to obtain an effective local concentration of antibiotic in the ear. Another object of the invention is the provision of effective local concentrations of an analgesic to the affected ear of a patient suffering from otitis.

Accordingly, in one aspect, the present invention provides a method for preventing or reducing ototoxicity in a patient undergoing treatment with an aminoglycoside antibiotic, comprising administering to the patient a locally effective amount of an otoprotective agent.

In another aspect, the present invention provides a method for preventing or reducing ototoxicity in a patient undergoing treatment with a loop diuretic agent.

In yet a further aspect, the present invention provides a method for preventing or reducing ototoxicity in a patient undergoing treatment with quinine or quinidine for conditions in which such compounds are indicated.

In another aspect, the present invention provides a method for preventing or reducing ototoxicity in a patient exposed to noise for a time and at an intensity sufficient to result in ototoxicity.

The invention provides sustained-release devices, adapted for insertion into an inner ear cavity, for administration of ototoprotective agents, as well as a method of reducing the ototoxic effect of a chemotherapeutic agent upon a subject which comprises inserting into an inner ear cavity of the subject a sustained-release device of the invention.

Furthermore, an improvement in the present invention relates to methods for augmenting treatments which require administration of a chemotherapeutic agent that has an ototoxic and hearing-impairing side effect. The improvement includes administering prophylactically or therapeutically an effective amount of an otoprotective agent to prevent, reduce or treat the ototoxic side effects of the chemotherapeutic drug without impairing its efficacy. The otoprotective agent and chemotherapeutic agent may be provided in various modes including administration prior to, simultaneously with, or subsequent to administration of said ototoxic chemotherapeutic agent. The otoprotective agent and chemotherapeutic agent may also be provided in various forms including but not limited to a single pharmaceutical preparation, e.g., as a single dosage form, or a kit in which each is provided in separate dosages, along with instructions for co-administering the two agents.

Another aspect of the invention provides a method for treating a mammal to prevent, reduce, or treat a hearing impairment, disorder or imbalance, including but not limited to ototoxin-induced hearing impairment, by administering to a mammal in need of such treatment an otoprotective agent formulated in a sustained release device. One embodiment is a method for treating a hearing disorder or impairment wherein the ototoxicity results from administration of a therapeutically effective amount of an ototoxic pharmaceutical drug. Typical ototoxic drugs include but are not limited to chemotherapeutic agents, e.g. antineoplastic agents, and antibiotics. Other ototoxic drugs include loop diuretics, quinines or a quinine-like compound, and salicylate or salicylate-like compounds.

The methods of the invention are effective when the ototoxic compound is an antibiotic, preferably an aminoglycoside, macrolide, or glycopeptide antibiotic. Ototoxic aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include neomycin B, kanamycin A, kanamycin B, gentamicin C1, gentamicin C1a, and gentamicin C2. Ototoxic macrolide antibiotics include but are not limited to erythromycin and azithromycin. Glycopeptide antibiotics include but are not limited to vancomycin.

Hearing impairments induced by aminoglycosides can be prevented or reduced by the methods of the invention. Although the aminoglycosides are particularly useful due to their rapid bactericidal action in infections by susceptible organisms, their use is limited to more severe, complicated infections because of ototoxic and nephrotoxic side-effects. For this reason the aminoglycosides are considered to have a low therapeutic/risk ratio compared to other antibiotics used systemically.

The aminoglycoside antibiotics which can be employed in conjunction with the ototoxicity inhibiting compositions of the invention may be any aminoglycoside antibiotic. Examples of such aminoglycoside antibiotics include but are not limited to kanamycin, gentamicin, amikacin), dibekacin, tobramycin, streptomycin, paromomycin, sisomicin, isepamicin, and netilmicin, all known in the art. Other useful antibiotics include the many structural variants of the above compounds (e.g. kanamycins A, B and C; gentamicins A, C1, C1, C2 and D; neomycins B and C, and the like).

Accordingly, the methods and compositions of the invention find use for the prevention and treatment of opportunistic infections in animals, including man. Compositions and methods of the invention may be used advantageously in combination with known antimicrobial agents to provide improved methods for prevention and treatment diseases induced by Gram-positive, Gram-negative, and acid-fast bacteria. Use of a composition of the invention in combination with such agents permits a higher dosage of the antimicrobial agents, increasing therapeutic (antibacterial) effectiveness without increasing the risk of ototoxic side effects.

The present invention also provides methods for conducting pharmaceutical business, which involve one or more of manufacturing, testing, marketing, distributing, and licensing preparations or kits for co-administering an otoprotective agent with an ototoxic chemotherapeutic agent.

### BRIEF DESCRIPTION OF THE FIGURES

FIG. 1 shows the time course of release of cyclosporin A into a phosphate buffer at pH 7.4 from a disc-shaped sustained-release device 2.5 mm in diameter. Error bars represent standard deviation from the mean.

FIG. 2 shows the time course of release of fluocinolone acetonide into a phosphate buffer at pH 7.4 from a rod-shaped sustained-release device 1 mm in diameter and 2-3 mm in length. Error bars represent standard deviation from the mean.

### DETAILED DESCRIPTION OF THE INVENTION

Otoprotective agents would be useful in the context of coping with the hazards to hearing posed by loud noises in certain occupational or recreational activities, or injuries arising from aging or exposure to ototoxic chemicals, if they could be delivered consistently to the inner ear at effective concentrations. The invention provides methods for using such otoprotective agents, which are useful for counteracting the ototoxic side-effects associated with certain chemotherapeutic regimes, and for improving quality of life in aging populations experiencing progressive hearing impairment.

One aspect of the invention is a method for preventing, reducing or treating ototoxicity in a subject undergoing treatment with an ototoxic chemotherapeutic drug, such as one selected from an aminoglycoside antibiotic, a macrolide antibiotic, a glycopeptide antibiotic, a platinum-containing antineoplastic agent, certain quinine-like compounds or an ototoxic loop diuretic drug, by implanting into the ear of a subject in need of such treatment a sustained-release drug delivery device capable of delivering a therapeutic dosage of an otoprotective agent, as disclosed further herein. Methods of implanting electrodes and other intra-cochlear devices are known in the art, as are methods of introducing solutions via cannulas and needles, and these methods may be readily adapted for implantation of the sustained-delivery devices of the invention.

Another aspect of the present invention relates to methods for augmenting treatments which require administration of an ototoxic chemical or chemotherapeutic agent comprising of administering an effective amount of an otoprotective agent to prevent, reduce or treat the hearing impairment caused by the ototoxic agent. In certain embodiments, the otoprotective agent and chemotherapeutic agent may be provided as a kit in which each is provided in appropriate dosage forms, along with instructions for co-administering the two agents.

In one embodiment, the device may be implanted prior to, simultaneously with, or subsequent to administration of said ototoxic chemotherapeutic agent.

In a certain embodiment, the invention provides a method wherein a therapeutically effective amount of otoprotective composition is administered to prevent, reduce, or otherwise treat hearing impairment due to NIHL, wherein the otoprotective agent is administered between 72 hours before, and 36 hours after exposure to otodestructive noise. The preferred timing of administration will be dependent on the times during which the otoprotective agent exhibits the desired otoprotective effects.

In other embodiments, the invention provides methods and compositions for delivering therapeutic drugs to the inner ear, such as antibiotics, neurologically active agents, growth factors, and the like.

Representative aminoglycoside antibiotics include, but are not limited to, amikacin (BB-K8), butirosin, geneticin, gentamicin, kanamycin, lividomycin, neomycin, paromomycin, hybrimycin, propikacin (UK 31214), ribostamycin, seldomycin, trehalosamine, □-D-mannosyl-□-D-glucosaminide, apramycin, bluensomycin, netromycin, streptomycin, tobramycin, sisomicin, destomycin, Antibiotic A-396-I, dibekacin, kasugamycin, fortimicin, or derivatives, analogs or variants thereof. Representative macrolide antibiotics include, but are not limited to, erythromycin and azithromycin, and a representative glycopeptide antibiotic is vancomycin.

Representative platinum-containing antineoplastic agents include, but are not limited to, cis-diaminedichloroplatinum(II) (cisplatin), trans-diaminedichloroplatinum(II), cis-diamine-diaquaplatinum(II)-ion, chloro(diethylenetriamine)-platinum(II) chloride, dichloro(ethylene-diamine)-platinum(II), diamine(1,1-cyclobutanedi-carboxylato)-platinum(II), spiroplatin, dichlorotrans-dihydroxybisisopropolamine platinum IV (iproplatin), diamine(2-ethylmalonato)-platinum(II), ethylenediamine-malonatoplatinum(II), aqua(1,2-diaminodyclohexane)-sulfatoplatinum(II), (1,2-diaminocyclohexane)malonato-platinum(II), (4-carboxyphthalato)(1,2-diaminocyclo-hexane)-platinum(II), (1,2-diaminocyclohexane)-(isocitrato)platinum(II), (1,2-diaminocyclohexane)-cis(pyruvato)platinum(II), or (1,2-diaminocyclohexane)-oxalatoplatinum(II).

Another aspect of the invention is a pharmaceutical dosage form comprising a sustained-release device adapted to deliver to the inner ear a therapeutically effective amount of an otoprotective compound, or a pharmaceutically acceptable salt, tautomer solvate, clathrate, prodrug or metabolic derivative thereof.

In general, the therapeutically effective amount of the pharmaceutical dosage will be in the range of from about 0.1 μg per gram of treated tissue to about 500 μg per gram. In specific embodiments it may range from about 0.1 μg per gram to about 200 μg per gram, in still other embodiments it may be from about 100 μg per gram to about 500 μg per gram. These amounts are expressed in terms of local effective concentrations within the treated ear tissue, and it should be understood that the concentrations will range from a relatively high level immediately adjacent to the implanted device or composition, to insignificant levels in distant tissues.

Another aspect of the present invention is a method for conducting a pharmaceutical business, comprising:

(a) providing an otoprotective agent in the form of a sustained release device, optionally in the form of a kit comprising said sustained release device provided conjointly with an ototoxic chemotherapeutic drug; and

(b) advertising to healthcare providers the benefits of using the sustained release device or kit as a means of reducing the ototoxic side-effects associated with said ototoxic chemotherapeutic drug.

In one embodiment, the method may further comprise the steps of:

(a) providing a distribution network for selling the device or kit; and

(b) providing instructions to patients or physicians for using the device or kit to reduce said ototoxic side-effects.

The present invention provides another method for conducting a pharmaceutical business, comprising:

(a) for a selected ototoxic chemotherapeutic agent, determining effective formulations and dosages for an otoprotective agent in the form of a sustained release device to be co-administered with the ototoxic chemotherapeutic agent;

(b) conducting safety and efficacy profiling of the sustained release device having formulations and dosages determined in step (a) when co-administered with the selected ototoxic chemotherapeutic agent, and

(c) providing a distribution network for selling a sustained release device having the formulation and dosage identified in step (b) as having an acceptable therapeutic profile.

In the above embodiment, step (b) may optionally involve licensing to another business entity the rights for further development of a sustained release device having the formulation and dosage identified in step (a).

In any of the above embodiments, step (c) may optionally involve licensing to another business entity the rights for distribution and sale of a preparation identified in step (b) as having an acceptable therapeutic profile.

The present invention also relates to methods useful for treating a patient for disorders of the ear or its adjacent structures, and more particularly treating otic disorders in mammals.

Diseases of the ear are categorized into diseases of external, middle and inner ear. One symptom common to all of these conditions is hearing loss. Hearing loss is characterized as conductive or sensorineuronal loss. Conductive loss is a rare condition, except for glomus jugulare tumors and neuromas of the seventh nerve with extension into the middle ear. Sensorineuronal loss can be further subdivided into neuronal or retrocochlear and sensory or cochlear losses. Causes of neuronal or retrocochlear hearing loss include acoustic neuroma or cerebellopontine angle lesions. With rare exceptions, neurotologic diseases cause a sensorineuronal type of hearing loss. The characteristics of a cochlear loss, however, reflects hair cell damage with an intact eight nerve. Common causes of cochlear hearing loss include sudden hearing loss, ototoxicity, noise-induced hearing loss, congenital and early onset hearing loss, presbycusis, and metabolic causes.

Hearing impairments relevant to the invention are preferably sensory hearing loss due to end-organ lesions involving inner ear hair cells, e.g., acoustic trauma, viral endolymphatic labyrinthitis, Meniere's disease. Hearing impairments include tinnitus, which is a perception of sound in the absence of an acoustic stimulus, and may be intermittent or continuous, wherein there is diagnosed a sensorineural loss. Hearing loss may be due to bacterial or viral infection, such as in herpes zoster oticus, purulent labyrinthitis arising from acute otitis media, purulent meningitis, chronic otitis media, sudden deafness including that of viral origin, e.g., viral endolymphatic labyrinthitis caused by viruses including mumps, measles, influenza, chickenpox, mononucleosis and adenoviruses. The hearing loss can be congenital, such as that caused by rubella, anoxia during birth, bleeding into the inner ear due to trauma during delivery, ototoxic drugs administered to the mother, erythroblastosis fetalis, and hereditary conditions including Waardenburg's syndrome and Hurler's syndrome. The hearing loss can be noise-induced, generally due to a noise greater than 85 decibels (db) that damages the inner ear. Hearing loss includes presbycusis, which is a sensorineural hearing loss occurring as a normal part of aging, fractures of the temporal bone extending into the middle ear and rupturing the tympanic membrane and possibly the ossicular chain, fractures affecting the cochlea, and acoustic neurinoma, which are tumors generally of Schwann cell origin that arise from either the auditory or vestibular divisions of the 8th nerve. In particular, the hearing loss may be caused by an ototoxic drug that effects the auditory portion of the inner ear, particularly inner ear hair cells. More detailed information about the etiology of hearing loss can be found in Chapters 196, 197, 198 and 199 of The Merck Manual of Diagnosis and Therapy, 14th Edition, (1982), Merck Sharp & Dome Research Laboratories, N.J. and corresponding chapters in the most recent 16th edition, including Chapters 207 and 210) relating to description and diagnosis of hearing and balance impairments. These chapters are incorporated by reference herein.

Another group of disorders are noise-induced hearing loss and presbycusis (hearing loss due to aging). Some of the recognized factors involved in these types of hearing loss are genetic, vascular, noise, dietary, hypertension, and metabolic causes. This occurs due to a gradual, usually symmetrical loss of sensory hair loss and nerve fibers. The degeneration is initially sensory and the neural degeneration is presumably secondary. Typical pharmaceutical compounds that may be useful to treat these conditions include, but are not limited to, calcium channel blocking agents, immunosuppressants such as cyclosporins, neuromodulators, steroids, and growth factors such as IGF-1 and FGF-2.

Another group of disorders which may cause sensorineuronal hearing loss, and which are treatable by the methods, compositions, and devices of the invention, are the peripheral vestibular disorders. The peripheral vestibular system consists of the vestibular portion of cranial nerve (CN) VIII and the balance organs of the inner ear: the utricle, the saccule, and the semicircular canals. Lesions of these organs affect the balance function and cause vertigo and disequilibrium. Some of the disorders may be associated with various degrees and combinations of hearing loss, tinnitus, hyperacusis, or diplacusis. Peripheral vestibular disorders are subdivided into primary and secondary causes or lesions. Primary lesions begin in and are limited to the inner ear or vestibular nerve. Secondary lesions begin elsewhere, such as in the middle ear or cranial base, and progress to involve the inner ear.

One peripheral vestibular disorders with primary lesion is Endolymphatic Hydrops which includes Meniere's disease. Endolymphatic hydrops is a condition of the inner ear that has many different causes. When a specific cause cannot be identified, the condition is termed Meniere's disease. Endolymphatic hydrops is characterized by distention and distortion of the endolymph-containing structures of the labyrinth. Hydrops usually manifests as episodic vertigo, fluctuating sensory hearing loss, tinnitus, and aural fullness. Some known causes of endolymphatic hydrops are acoustic trauma, autoimmune inner ear disease, chronic otitis media, Cogan's syndrome, congenital deafness fenestration of the otic capsule, labyrinthine concussion, Letterer-Siwe disease, leukemia, Lindau-von Hippel disease, Mondini dysplasia, otosclerosis, Paget's disease, serous labyrinthitis, surgical inner ear trauma, syphilis, temporal bone trauma, and viral labyrinthitis.

Meniere's disease (idiopathic endolymphatic hydrops) is characterized by an episodic abnormal sensation of movement when there is no motion or an exaggerated sense of motion in response to a given bodily movement (vertigo), progressive loss of hearing in one or both ears, and abnormal noises or ringing in the ear (tinnitus). The fluid-filled semicircular canals (“labyrinth”) of the inner ear, along with the eighth cranial nerve, control balance and position sense. Meniere's disease involves a swelling of the part of the canal (endolymphatic sac) that controls the filtration and excretion of the fluid of the semicircular canal. Some risk factors for developing Meniere's disease include recent viral illness, respiratory infection, stress, fatigue, use of prescription or nonprescription drugs including aspirin, and a history of allergies, smoking, and alcohol use.

While prompt treatment of an ear infection and other related disorders may help prevent Meniere's disease, there remains a need for a more targeted therapy. As there is no known cure for Meniere's disease, treatment has focused on relieving symptoms by lowering the pressure within the endolymphatic sac. Therefore, treatment for Meniere's disease is generally directed at reducing inner ear fluid volume, increasing inner ear blood circulation, and/or arresting the effect of immune reactivity or hydropic damage that has occurred.

Long term therapy for hydrops aims to decrease inner ear fluid volume by dietary sodium restriction and diuresis. Diuresis is achieved by combination therapy with antidiuretics such as thiazide, triamterene, or carbonic anhydrase. Associated side effects include hypokalemia. Vasodilators have also been used in treating Meniere's disease. Betahistine, niacin, and papaverine are some vasodilators that have been employed with limited success.

Vestibular suppressant medications are another group of drugs that have been used in controlling vertigo in peripheral vestibular disorders. These drugs have variable anticholinergic, anitemetic, and sedative properties. Diazepam, meclizine, dimenhydrinate, prochlorperazine, promethazine, and preazepam are some examples of this group of drugs. In some rare cases when the patient's vertigo is uncontrollable, hospitalization may be necessary. In such cases, intravenous or intramuscular fentanyl citrate and droperidol are very effective. However, these drugs are potent respiratory depressants and their systemic administration must be closely monitored.

In addition to vestibular suppressants several anticholinergic medications may occasionally be useful in managing Meniere's patients. Glycopyrrolate, propantheline, and atropine can be effective in mitigating nausea and atypical or minor forms of vertigo. Scopolamine is useful in ameliorating motion sickness.

Corticosteroids have also been used to limit the inflammatory response. Steroids such as dexamethosone or prednisone can often effect a reversal in the sudden hearing loss that occurs sometimes after months or years of symptom free hydrops patients. Steroids are co-administered with antacids and H2 Blocker to counter their side effects.

Another mode of treating Meniere's disease is unilateral chemical vestibular ablation. The use of ototoxic drugs such as aminoglycosides installed into the tympanic cavity has been used to treat unilateral Meniere's disease. Severe hearing loss is usually the side effect of this treatment.

Use of many of these medications is limited due to severe side effects associated with systemic administration. Systemic administration of cyclophosphamide, for example, for treatment of autoimmune autologic dysfunction leads to manifestation of neutropenia. Additionally, this drug is contraindicated in treating patients with a history of bleeding ulcers or poorly controlled insulin-dependent diabetes. Diuretics, which are the mainstay of treating conditions associated with hydrops, may cause hyperkalemia which is associated with muscle cramps, weakness, lassitude, and some cardiac arrhythmias.

Surgical treatment of Meniere's disease most certainly relieves vertigo symptoms by totally ablating the erratically reacting labyrinth, but entails complete loss of hearing in the affected ear. Conservative surgical approaches which attempt to conserve auditory functions while treat vertigo symptoms include endolymphatic sac decompensation, cochleostomy, cochlear dialysis, sacculotomy, grommet insertion, cervical sympathectomy, vestibular nerve division, ultrasonic destruction of the vestibular labyrinth, and interatympanic injection of ototoxic drugs. Radical surgical approach involves the total destruction of the membranous labyrinth. A surgical approach is oftentimes contraindicated for the high incidence of complete hearing loss in the ear caused by surgically opening the inner ear.

Therefore, there still exists a need for treating conditions such as Meniere's disease and those associated with cochlear hair cell loss, where more effective methods are employed other than dietary precautions, systemic drug administration, or surgically opening the inner ear.

Exemplary medicines which are typically used to treat inner ear tissues include but are not limited to urea, mannitol, sorbitol, glycerol, lidocaine, xylocaine, epinephrine, immunoglobulins, sodium chloride, steroids, heparin, hyaluronidase, aminoglycoside antibiotics (streptomycin/gentamycin), and other drugs, biological materials, and pharmaceutical compositions suitable for treating tissues of the human body. Likewise, treatment of inner ear tissues and/or fluids may involve altering the pressure, volumetric, and temperature characteristics thereof. Imbalances in the pressure levels of such fluids can cause various problems, including but not limited to conditions known as endolymphatic hydrops, endolymphatic hypertension, perilymphatic hypertension, and perilymphatic hydrops.

Due to the risks that certain drugs impose, researchers have developed systems for administering such drugs to aid in the treatment of these ailments and diseases. Many of these systems provide a release rate which reduces the occurrence of detrimental side effects.

Currently, there are several procedures available to locally deliver medication to the inner ear. In one procedure, a physician injects medication into the middle ear over the round window area through the tympanic membrane. The patient is asked not to swallow and must remain relatively still in the supine position with the head turned both during the injection and for some time afterwards (at least 30 minutes) to allow the medication to diffuse through the round window. A tube could be placed in the ear drum to convey medication. As there is no pathway for evacuation of air as the medication is applied through the tube, it is difficult to get the medication to go into the middle ear. The situation is analogous to an attempt to pour fluid into a container that has only one hole. If the medication does not get into the middle ear, it may flow down the eustachian tube or it may not get directly to the round window membrane. In fact, most medication is lost down the eustachian tube when the patient swallows. Another drawback to this procedure is the need for repeated doctor visits for injection of the medicament. As a result, this procedure is not very cost- or time-efficient.

One technique for direct treatment that has been developed uses a small gelatin sponge placed on the round window membrane. The physician then injects the medication directly onto the sponge. Like direct treatment with a tube, this procedure requires frequent administrations, and the medicament may be lost down the eustachian tube. The rate of drug release from the sponge is not well-controlled, and furthermore, the gelatin material that the sponge is made of may deteriorate.

Another technique utilizes an indwelling catheter that requires an operating room surgical procedure for implantation. A micro-pump can be attached to the catheter to deliver exact amounts of medication. Although this technique has had some clinical success, the catheter, micro-pump, surgical procedure, and subsequent hospitalization are very expensive, and implantation involves a surgical procedure with its attendant risks.

U.S. Pat. No. 6,120,484, to Silverstein, describes a device for administering a drug to the ear. The device is a wick-like otological implant for delivery of medicament to a treatment site in the inner ear. The device is made of a material capable of conveying the medicament by capillary action from the outer ear, through the eardrum and onto the surface of the round window of the inner ear. The method relies upon subsequent diffusion of the medicament through the round window membrane and into the inner ear.

Another type of device used for sustained release of a drug to the ear, described in U.S. Pat. No. 5,895,372, to Zenner et al, is an implantable dosaging system for medications, active substances, etc., for administration in a form of dissolved or suspended fluids, using a pump mechanism. This device includes a medication reservoir equipped with a sealing injection port connected to it, and a pump located within the reservoir for pumping a medication out through a discharge opening. There is also a pump outlet for administering the medication. This device is transcutaneously operable with the aid of an actuator located on the reservoir. The medication reservoir is made from biocompatible plastic material and is intended for implantation by fixing to the muscular fascia, for example, in the vicinity of the axilla, neck, or occiput.

Another treatment system, described in U.S. Pat. No. 5,474,529, is an apparatus for use in the middle and inner ear using a diffusion mechanism. The apparatus includes a tubular stem portion attached to a medicine-retaining reservoir with an internal cavity. The reservoir includes multiple pores and openings having semipermeable membrane which enables medicine delivery from the reservoir. Delivery occurs when the reservoir comes in contact with selected middle-inner ear interface tissues. A conductive member for receiving electrical potentials from ear tissues is affixed to the apparatus. Alternatively, the apparatus may include two tubular stem portions secured on opposite sides of a reservoir along with a conductive member attached thereto of the type indicated above. This apparatus is surgically inserted so that the first tubular stem portion is placed within the inner ear. At least part of the apparatus (the second tubular stem portion) resides within the external auditory canal. This apparatus is purported in the patent to deliver medications into the middle or inner ear.

The above described systems and devices are intended to provide sustained release of drugs for obtaining desired physiological or pharmacological effects. However, there are disadvantages associated with their use, including the fact that it is often difficult to obtain the desired release rate and the desired concentration of the drug. This difficulty is largely due to the variability of drug release from the devices, combined with the poorly predictable rate of diffusion of the drug into the inner ear and a dependence upon the precise placement of the device.

The present invention employs an implanted sustained-release drug device, as described herein, which overcomes these disadvantages. In one embodiment of the invention, the device includes an inner core or reservoir including the effective medicament, an impermeable tube which encloses a portion of the reservoir, and a permeable member, preferably at an end of the tube, through which the medicament diffuses into the surrounding medium. Such a device is effective in delivering an effective and sustained concentration of a medicament to the inner ear, thereby obtaining a desired local physiological or pharmacological effect without the complications of systemic administration.

In one embodiment, the device is a rod shaped device containing a drug core in a polymer-drug matrix form, which is preferably surrounded by one or more layers of polymer, at least one of which is permeable to the drug. The polymer layers may be applied to the core, or the core may be formed within a pre-manufactured sheath. The size of the device is preferably about 1.0 mm in diameter and 2.0 to 3.0 mm in length. This device provides a zero order release profile in vitro over a prolonged time period, as shown in FIG. 2 for a device having a fluocinolone acetonide core. Drug-polymer matrices suitable for use in the core of the device are known, as disclosed for example in international patent application WO 02/087586. Devices of this configuration are known in the art, as disclosed for example in U.S. Pat. No. 6,375,972.

It has been found that by sealing at least one surface of a cylindrical device with an impermeable member which is capable of supporting its own weight, which has dimensional stability, and/or which has the ability to accept a drug core therein without changing shape, manufacture of the entire device is made simpler. That is, the use of a pre-manufactured sheath or tube of material to hold the drug reservoir during manufacture allows for significantly easier handling of the tube and reservoir, because unlike a coating, a tube can fully support both its own weight and the weight of the reservoir. Also, this rigid structure allows the use of drug slurries drawn into the tube, which allows the fabrication of longer cylindrical devices. Furthermore, because of the relative ease of manufacturing these devices, more than one reservoir, optionally containing more than one drug, can be incorporated into a single device.

In another embodiment, the device is approximately spherical in shape, and comprises a round pellet or core of a drug or drug-polymer matrix, preferably surrounded by at least one polymer layer through which the drug diffuses. This embodiment is particularly suitable for implantation via cannula or needle.

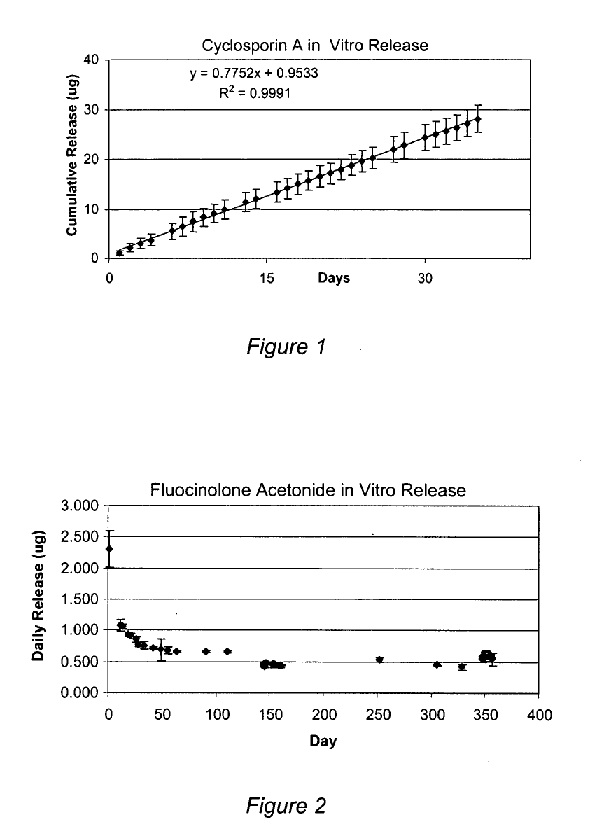

In another embodiment, the invention employs a disc shaped device containing a drug core and having one or more diffusion ports. The disc is preferably coated with one or more layers, at least one of which is permeable to the drug. The device is preferably about 2 mm in diameter and about 2 mm thick, and is more preferably smaller. Such a device provides a zero order release profile in vitro over a prolonged time period, as shown in FIG. 1 for a device with a core of cyclosporin A. Such devices are known in the art, and have been disclosed in U.S. Pat. No. 5,902,598.

Thus, the present invention provides a method for the placement, controlled and sustained release of a composition effective in obtaining a desired local or systemic physiological or pharmacological effect.

In one embodiment the invention constitutes a method for treating a mammal having or prone to a hearing (or balance) impairment or treating a mammal prophylactically to prevent or reduce the occurrence or severity of a hearing (or balance) impairment that would result from inner ear cell injury, loss, or degeneration, preferably caused by an ototoxic agent, wherein a therapeutically effective amount of an otoprotective agent is introduced.

The method includes positioning a sustained released drug delivery system at an area wherein release of the agent is desired and allowing the agent to pass through the device to the desired area of treatment.

The invention provides a method for direct implantation of a drug delivery device in to the inner ear in the vicinity of the oval window. Such devices provide sustained controlled release of various compositions to treat the inner ear without risk of detrimental local and systemic side effects. Preferably such devices use a diffusion mechanism in delivery of the agents to the treatment area. The device preferably maintains an effective concentration of the drug for at least 30 days, more preferably 180 days, and most preferably for at least one year.

Accordingly an aspect of the invention is a method of treating a condition of the ear of a mammal comprising the steps of accessing an internal anatomical site adjacent to the inner ear, and placing or implanting a drug delivery device in the internal anatomical site.

Still other aspects, features, and attendant advantages of the present invention will become apparent to those skilled in the art from a reading of the following detailed description of embodiments constructed in accordance therewith, taken in conjunction with the accompanying drawings.

More specifically, the present inventors have discovered a method that is suitable for the placement, controlled and sustained release of an agent or drug effective in obtaining a desired local physiological or pharmacological effect.

Another aspect of the present invention is a method for effectively and safely delivering an effective amount of therapeutic agents, including co-drugs. Co-drugs are described in U.S. Pat. No. 6,051,576 to Ashton, et al., the entirety of which is incorporated by reference herein.

One embodiment of the present invention is single drug or co-drug of one or more pharmacologically active compounds in the following classes of agents: anti-inflammatory and analgesic agents, including but not limited to fentanyl citrate; non-steroidal anti-inflammatory (NSAID) agents, including but not limited to salicylates; tranquilizing agents, including but not limited to droperidol and prochlorperazine; corticosteroids, including but not limited to flucinolone, dexamethasone and prednisone; volume expanding agents; vasodilating agents, including but not limited to batahistine, niacin and papaverine; antihistaminic agents, including but not limited to meclizine, dimenhydrinate, scopolamene, and promethazine; anticholinergic agents, including but not limited to glycopyrrolate, propantheline, and atropine; antibiotic agents, including but not limited to ampicillin, cefuroxime, amoxicillin and ceftriaxone; antiviral agents; immunosuppressive agents, including but not limited to cyclophosphomide and cyclosporine; diuretic agents, including but not limited to thiazide, triamterene and carbonic anhydrase inhibitors; antacids and H2-blockers, including but not limited to nizatidine and cimetidine; antiemetics, including but not limited to metoclopramide or diphenidol; calcium channel blockers, including but not limited to diltiazem, nifedipine and verapamil; anticancer agents and drugs; vitamins; vascular rheologic agents; neuroprotective agents; neuromodulators; and anti-apoptotic agents.

Co-drugs in the present invention may include one or more drugs combined as described in U.S. Pat. No. 6,051,576, and below. Co-drugs in the present invention also includes co-drug of a single compound (i.e, a co-drug in which the two active components are the same agent). Those of skill in the art will readily appreciate that the present invention is not limited to the specific agents listed herein, but extends to compounds with desirable therapeutic effects and/or for which the use is indicated for the particular disease state of interest. More detailed lists of the therapeutic agents to which the present invention can be found in, e.g., Goodman & Gilman's The Pharmacologic Basis of Therapeutics (10th ed., McGraw-Hill Companies, Inc., 2001), Remington's Pharmaceutical Sciences (18th ed., Mack Publishing Co., 1990), The Merck Index (12th ed., Merck Research Laboratories, 1996), and other such volumes.

The present invention includes implanting drug delivery devices to deliver therapeutic agents, as described in this application, to a localized anatomical site within the ear. Numerous drug delivery devices are usable in the present invention, for example the devices described in U.S. Pat. No. 5,378,475, to Smith et al; U.S. Pat. No. 5,836,935, to Ashton et al.; U.S. Pat. No. 5,902,598, to Chen et al.; and U.S. Pat. No. 6,375,972, to Hong Guo et al. The entire contents of each of these patents is incorporated by reference herein. When a method in accordance with the present invention necessitates the use of more than one such device, either for delivery of more than one medicament or in order to deliver sufficient medicament, another aspect of the present invention is using two or more drug delivery devices, which may be the same or different. It will be appreciated that the devices described, in order to be useful in the present invention, must be adapted for insertion into an inner ear cavity as described elsewhere in this disclosure.

A preferred embodiment of the present invention is a method for safely delivering an effective amounts of a therapeutic agent, or a pro-drug or co-drug, by inserting into an inner ear cavity an implantable drug delivery device. The device preferably functions by a diffusion mechanism.

A particularly preferred embodiment of the present invention is a method for delivering an effective amount of therapeutic agents, including co-drugs and pro-drugs, using rod-shaped implantable drug delivery devices as described in U.S. Pat. No. 6,375,972.

Yet another preferred embodiment of the present invention is a method for delivering, for an extended period of time, an effective amount of therapeutic agents to an affected site. Long term delivery of therapeutic agents is a preferred embodiment of the present invention. Therefore, the present invention includes a drug delivery device that is placed within an inner ear cavity and is capable of delivering a therapeutic agent for at least a week. Preferably the duration of the drug delivery through the implanted drug delivery device to the effected site is months to years. More preferably the delivery of these therapeutic agents is linear in nature and the dosage is capable of remaining at therapeutic levels for weeks, months, or years.

There are several aspects to the present invention. In general, one aspect of the present invention is the treatment of conditions associated with the ear by avoiding systemic administration and delivery of active medication, to thereby reduce, minimize, or eliminate the associated side effects. Therefore, an aspect of the present invention is the localized delivery of medication to the ear, including the inner ear, using a drug delivery device which is implantable.

Specifically, the present invention provides a method for treating inner ear diseases and their associated symptoms including, but not limited to, congenital abnormalities such as congenital syphilis and toxoplasmosis; viral or bacterial infections; cancers; and acquired inner ear diseases such as Meniere's disease, sensory neuronal hearing loss or ototoxicity. Another aspect involves maintaining the integrity or keeping cochlear hair cells intact within the inner ear. The goal is, therefore, to leave vestibular hair cells intact. Thus, it would be advantageous to administer gentamicin to a patient via a local route of administration and thereby avoid undesirable side effects of systemic administration.

More particularly, senility- and noise-induced loss of hearing can be treated according to the present invention. It is known that there is an apoptosis of hair cells within the cochlear ear channels associated with some of these conditions. According to the present invention, this condition may be treated by administering drugs directly to the inner ear in order to minimize or delay this senility- or noise-induced hearing loss. Typical pharmaceutical compounds that may be useful include the calcium channel blocking agents, cyclosporins, as well as steroids.

Devices and methods in accordance with the present invention can also advantageously be used in the treatment of Meniere's disease. Preferred medications which may be used in treating this disease are mentioned above and include, but not limited to, vasodilators, diuretics and steroids.

Devices and methods in accordance with the present invention can also advantageously allow for the gradual diffusion of medication across a membrane or into, e.g., the endolymphatic sac. By way of example and not of limitation, a hole is drilled into the endolymphatic sac or directly into the bone, and an implantable drug delivery device is secured into the resulting hole. The device may be screw-shaped or otherwise shaped so as to be self-anchoring, or it may be attached by sutures, screws, staples, or other methods known in the art. At the tip of the screw may be a permeable polymer that modulates delivery of the drug in a controlled manner. According to another aspect of the present invention, the implantable drug delivery device can be implanted in the oval window or round window, and the drug from the device can leach into the inner ear to treat the condition for which the drug is selected.

While less preferable, another aspect of the present invention is the surgical implantation of a drug delivery device, which includes larger scale cutting of the tissues of the patient in order to access the anatomical site in which the drug delivery device is to be implanted.

While the invention has been described in detail with reference to preferred embodiments thereof, it will be apparent to one skilled in the art that various changes can be made, and equivalents employed, without departing from the scope of the invention.

In the methods of preventing or reducing ototoxicity of the present invention, various parameters associated with the patient's hearing and vestibular systems can be tested by methods well known in the art to establish pretreatment baseline values. After administration of the methionine protective agent, and over the course of chemotherapy and afterwards, ototoxic effects can be monitored by conventional tests, and the results can be compared to those obtained prior to treatment to determine if any change has occurred. If any impairment is observed, the amount and/or time of administration of the protective agent administered in conjunction with subsequent doses of the platinum-containing chemotherapeutic agent, loop diuretic agent, aminoglycoside antibiotic, iron chelating agent, quinine, quinidine, or exposure to noise or radiation, can be adjusted so as to reduce or prevent further ototoxic changes without substantially diminishing the antineoplastic effectiveness of the platinum-containing chemotherapeutic agent or radiation, the diuretic effect of the loop diuretic agent, etc. Similar modification of treatment parameters in the case of weight loss, gastrointestinal toxicity due to either the platinum-containing chemotherapeutic agent or radiation, neurotoxicity due to either the platinum-containing chemotherapeutic agent or radiation, alopecia due to either the platinum-containing chemotherapeutic agent or radiation, and overall patient condition/survival due to either the platinum-containing chemotherapeutic agent or radiation can be employed to optimize the protective effects of the protective agent with respect thereto. This can be achieved via appropriate testing and comparison of pre- and post-treatment values, e.g., patient weight and patient physical/medical/physiological condition, etc., with protocol adjustments being made as needed.

### DEFINITIONS

The term “inner ear cavity” refers to any of the various compartments of the inner ear, particularly fluid-filled cavities such as the scala typani, scala vestibuli, endolymphatic sacs and duct, and vestibular labyrinth, all compartments and ducts containing or connecting with these components, and any soft tissue in contact with these components from which an otoprotective agent may directly diffuse into an inner ear compartment.

By “adapted for insertion into an inner ear cavity” is meant that the composition or device is of a size suitable for insertion into an inner ear cavity via a syringe, cannula, catheter, or similar device, and that surfaces which are exposed to body fluids and tissues are biocompatible. An appropriate size for a cylindrical device would be about one millimeter in diameter, preferably 0.75 millimeters, and most preferably 0.5 millimeters or less. The composition or device may be bioerodable, or it may resist bioerosion, in which case the composition or device may be designed for later removal, or it may be designed to remain in place indefinitely. A device according to the invention may be single-use, or optionally it may be designed to be re-filled at intervals with a therapeutic agent. It should be understood that all references to insertion of a device or composition are intended to apply to insertion of multiple individual devices or compositions. Instruments and methods suitable for inserting devices and medicaments into the inner ear are known in the art, as disclosed for example in U.S. Pat. Nos. 4,819,647, 5,476,446, 6,377,849, and 6,408,855.

The terms “sustained-release device” and “device” refer to any object which comprises a drug, pro-drug, or co-drug, and which is capable of releasing said drug, pro-drug, or co-drug at a steady rate over a prolonged period of time ranging from a week to a year or more, when implanted into a body. It includes erodable compositions, which may optionally be coated or encapsulated, and it also includes non-erodable reservoir devices, which may be single-use or refillable. The compositions and devices of the present invention that are suitable for insertion into an inner ear cavity include encapsulating devices, which are essentially containers for a medicament, wherein the medicament slowly diffuses through one or more openings or pores in the surface of the capsule, as well as devices where the medicament is actively dispensed, e.g through the actions of an osmotic or electromechanical pump. Also included are devices in which a medicament-containing core is surrounded entirely or in part by a permeable coating, through which the medicament gradually diffuses. Such devices may be manufactured for example by filling a pre-formed device, or by coating a pre-formed medicament core.

Also included are compositions and devices which gradually erode under the influence of bodily fluids and/or enzymes, and which release a medicament in the process. Such devices and compositions may contain the active medicament itself, or they may contain a relatively insoluble pro-drug which is gradually transformed via chemical or enzymatic action into the active medicament. The drug or pro-drug may be incorporated into an erodable polymer matrix. Also included are solid forms of relatively insoluble medicaments, which simply dissolve slowly over time. These various erodable and pro-drug compositions may be encapsulated or coated, as described above, in order to achieve the desired rate of release with a desired consistency. Numerous devices and compositions have been developed for insertion into other parts of the body, and it is anticipated that most of those that are capable of being manufactured at appropriately small dimensions (roughly 0.5 to 2 mm in diameter) may be adapted for insertion into an inner ear cavity.

The term “hearing loss” refers to both a complete loss of hearing due to noise, chemicals, infection, or age, or to a hearing impairment due to the aforementioned factors. The term “hearing impairment” refers to a diminished hearing capacity due to the aforementioned factors.

As used herein, the term “ototoxic” or “ototoxicity” includes, but is not limited to, any detrimental or pathologic change in the structure or function of the ear, including changes in hearing and balance. Auditory functional changes can include, but are not limited to, hearing loss or other changes in auditory threshold for any stimulus, perception of sound including recruitment (abnormal growth in the perception of loudness), ability to identify, localize, recognize, distinguish between, or process sounds, and/or distortion of sounds or any abnormality as identified by conventional auditory tests. This term also includes tinnitus (ringing or noises in the ear), which includes any perception of sound other than in response to an external signal. Further, ototoxicity includes any perceived or measured functional change in the balance or vestibular system, including, but not limited to, either induced or spontaneous vertigo, dysequilibrium, increased susceptibility to motion sickness, nausea, vomiting, nystagmus, syncope, lightheadedness, dizziness, difficulty in visual tracking secondary to vestibular or balance disorder or abnormality as measured on any test of vestibular or balance function. Structural changes can include any intra- or extra-cellular, multicellular, or organ change in the auditory or vestibular pathways from the external ear up through and including the cortex and all pathways in between.

By “ototoxic agent” in the context of the present invention is meant a substance that through its chemical action injures, impairs, or inhibits the activity of a component of the nervous system related to hearing, which in turn impairs hearing (and/or balance). In the context of the present invention, ototoxicity includes a deleterious effect on the inner ear hair cells. Ototoxic agents that cause hearing impairments include, but are not limited to, neoplastic agents such as vincristine, vinblastine, cisplatin, taxol, or dideoxy-compounds, e.g., dideoxyinosine; alcohol; metals; industrial toxins involved in occupational or environmental exposure; contaminants of food or medicinals; or over-doses of vitamins or therapeutic drugs, e.g., antibiotics such as penicillin or chloramphenicol, or megadoses of vitamins A, D, or B6, salicylates quinines and loop diuretics. Other toxic agents that can cause ototoxicity-inducing hearing impairment can be identified and characterized by methods as taught herein. Radiation is also an ototoxic agent for purposes of this disclosure.

By “exposure to an ototoxic agent” is meant that the ototoxic agent is made available to, or comes into contact with, a mammal. Exposure to an ototoxic agent can occur by direct administration, e.g., by ingestion or administration of a food, medicinal, or therapeutic agent, e.g., a chemotherapeutic agent, by accidental contamination, or by environmental exposure, e.g., aerial or aqueous exposure.

The term “otoprotective agent” refers to an agent that reduces, prevents, treats NIHL, CIHL or age induced hearing impairment, or prevents, ameliorates, or otherwise protects against otoxicity or hearing impairment.

The term “otodestructive” means that which causes hearing impairment.

The term “ototoxic chemotherapeutic drug” refers to a chemotherapeutic agent with an ototoxic, hearing-impairing side effect.

As used herein, “mammal” for purposes of treatment refers to any animal classified as a mammal, including humans, domestic, and farm animals, and zoo, sports, or pet animals, such as dogs, horses, cats, sheep, pigs, cows, etc. For the purposes of the present invention the preferred mammal is a human.

“Treatment” refers to both therapeutic treatment and prophylactic or preventative measures, wherein the object is to prevent or slow down (lessen) inner ear tissue-damage-related hearing disorder or impairment (or balance impairment), preferably ototoxin-induced or inducible, and involving inner ear hair cells. Those in need of treatment include those already experiencing a hearing impairment, those prone to having the impairment, and those in which the impairments are to be prevented. The hearing impairments are due to inner ear hair cell damage or loss, wherein the damage or loss is caused by infections, mechanical injury, loud sounds, aging, or, preferably, chemical-induced ototoxicity, wherein ototoxins include therapeutic drugs including antineoplastic agents, salicylates, quinines, and aminoglycoside antibiotics, contaminants in foods or medicinals, and environmental or industrial pollutants. Typically, treatment is performed to prevent or reduce ototoxicity, especially resulting from or expected to result from administration of therapeutic drugs. A therapeutically effective treatment according to the invention may be given immediately after the exposure to prevent or reduce the ototoxic effect. More preferably, treatment is provided prophylactically, either by administration prior to or concomitantly with the ototoxic pharmaceutical or the exposure to the ototoxin. The term “treatment” is intended to encompass prophylaxis, therapy and cure.

As used herein “chronic” refers to a disorder that is not acute but rather occurs more or less on a continuous level. A “disorder” is any condition that would benefit from treatment with the method, and compositions of the invention. The disorder being treated may be a combination of two or more of the above disorders, and may include auditory or vestibular neuron damage or loss.

As used herein, the term “preventing” means to reduce the risk of occurrence of an abnormal biological or a medical event, such as hearing loss, in a cell, a tissue, a system, animal or human.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms the disease, disorder or condition, i.e., causing regression of the disease, disorder and/or condition.

The term “as valence and stability permits” in reference to compounds disclosed herein refers to compounds that have in vitro or in vivo half-lives at room temperature of at least 12 hours, or at least 24 hours, and are preferably capable of being stored at 0° C. for a week without decomposing by more than about 10%.

The terms “half-life” or “half-lives” refer to the time required for half of a quantity of a substance to be converted to another chemically distinct species in vitro or in vivo.

The term “clathrate” refers to inclusion compounds in which the guest molecule is in a cage formed by the host molecule or by a lattice of host molecules.

The term “prodrug” refers to any compound that is converted to a more pharmacologically active compound under physiological conditions (i.e., in vivo). A common method for making a prodrug is to select moieties that are hydrolyzed under physiological conditions to provide the desired biologically active drug.

The term “metabolic derivative” refers to a compound derived by one or more in vitro or in vivo enzymatic transformations on the parent compound, wherein the resulting derivative has an ED₅₀ value as an otoprotective agent that is less than 1000 times the ED₅₀ value of the parent compound.

The term “aminoglycoside antibiotic” includes a broad class of amino sugar containing antibiotics well known in the art. The aminoglycoside agents described in the literature which are useful in the methods of the present invention include, but are not limited to, amikacin (BB-K8), butirosin, geneticin, gentamicin, kanamycin, lividomycin, neomycin, paromomycin, hybrimycin, propikacin (UK 31214), ribostamycin, seldomycin, trehalosamine, α-D-mannosyl-α-D-glucosaminide, apramycin, bluensomycin, netromycin, streptomycin, sisomicin, destomycin, antibiotic A-396-I, dibekacin, kasugamycin, fortimicin, netilmicin, hygromycin, and tobramycin, and derivatives, analogs or variants thereof. Also useful in the methods of the invention are ototoxic glycopeptide antibiotics such as vancomycin, and ototoxic macrolide antibiotics such as erythromycin.

The term “platinum-containing antineoplastic agents” includes a broad class of water-soluble, platinum coordination compounds well known in the art, typically having anti-tumor activity. The platinum-containing antineoplastic agents described in the literature which are useful in the methods of the present invention include, but are not limited to, cis-diaminedichloro-platinum(II) (cisplatin), trans-diaminedichloro-platinum(II), cis-diamine-diaquaplatinum(II)-ion, cis-diaminedichloroplatinum(II)-ion, chloro(diethylenetriamine)-platinum(II) chloride, dichloro(ethylenediamine)-platinum(II), diamine(1,1-cyclobutanedicarboxylato)-platinum(II) (carboplatin), spiroplatin, dichlorotrans-dihydroxybisisopropolamine platinum IV (iproplatin), diamine(2-ethylmalonato)platinum(II), ethylenediamine-malonatoplatinum(II), aqua(1,2-diaminodiclohexane)-sulfatoplatinum(II), (1,2-diaminocyclohexane)malonato-platinum(II), (4-carboxyphthalato)(1,2-diaminocyclo-hexane)-platinum(II), (1,2-diaminocyclohexane)-(isocitrato)platinum(II), (1,2-diaminocyclohexane)-cis(pyruvato)platinum(II), and (1,2-diaminocyclohexane)-oxalatoplatinum(II).

1-17. (canceled)

18. A sustained release drug delivery device for delivering a medicament to the middle or inner ear, and adapted for implantation into the middle or inner ear, wherein said device has a diameter of about 2.5 mm or less, a length of about 2 to 3 mm, and is capable of releasing said medicament at a rate which maintains a pharmacologically effective concentration of said medicament within the middle or inner ear.

19. A sustained-release drug delivery device according to claim 18, wherein the device is capable of maintaining a pharmacologically effective concentration of said medicament within the inner ear for a period of at least 30 days.

20. A sustained-release drug delivery device according to claim 18, wherein the device is capable of maintaining a pharmacologically effective concentration of said medicament within the inner ear for a period of at least 180 days.

21. A sustained-release drug delivery device according to claim 18, wherein the device is capable of maintaining a pharmacologically effective concentration of said medicament within the inner ear for a period of at least one year.

22-32. (canceled)

33. A sustained-release drug delivery device according to claim 18, wherein the medicament is an otoprotective agent.

34. A sustained-release drug delivery device according to claim 33, wherein the otoprotective agent is selected from IGF-1, FGF-2, aspirin, reduced glutathione, N-methyl-(D)-glucaminedithiocarbamate and (D)-methionine.

35. (canceled)

36. A packaged pharmaceutical product comprising the sustained release drug delivery device according to any one of claims 18-21, and 33-34, together with instructions for properly using the device in conjunction with administration of an ototoxic chemotherapeutic drug.

37-42. (canceled)

43. A sustained-release drug delivery device according to claim 18, wherein said device comprises a tube that contains a core or reservoir comprising said medicament.

44. A sustained-release drug delivery device according to claim 43, wherein said tube is impermeable.

45. A sustained-release drug delivery device according to claim 44, wherein at least one permeable layer is disposed over one or both ends of said tube.

46. A sustained-release drug delivery device according to claim 18, wherein the device has a diameter of about 1 mm.

47. A sustained-release drug delivery device according to claim 18, wherein the device has a diameter of about 0.75 mm.

48. A sustained-release drug delivery device according to claim 18, wherein the device has a diameter of about 0.5 mm or less.
